(19) United States
(12) Patent Application Publication
(10) Pub. No.: US 2007/0059370 A1
(43) Pub. Date: Mar. 15, 2007

(54) Method and apparatus for fabricating nanoparticles

(75) Inventors: Po-Fu Chou, Toyuan County (TW); Pei Kan, Hsinchu (TW); Chun Jung Chen, Yunlin (TW); Shih-Liang Yang, Kaohsiung (TW); Chun Fu Leu, Hsinchu (TW); Shin-San Su, Taipei (TW)

Correspondence Address: QUINTERO LAW OFFICE, PC, 2210 MAIN STREET, SUITE 200, SANTA MONICA, CA, 90405, US

(73) Assignee: INDUSTRIAL TECHNOLOGY RESEARCH INSTITUTE, HSINCHU (TW)

(21) Appl. No.: 11/229,129

(22) Filed: Sep. 15, 2005

(51) Int. Cl. A61K 9/14; B29B 9/00

(52) U.S. Cl. 424/489; 264/5; 977/906

(57) ABSTRACT

An apparatus and method for forming nanoparticles employs an inkjet dispenser and a nanoparticle formation device. The inkjet dispenser includes at least one orifice. A liquid solution with a substance to be transformed into nanoscale is received in the inkjet dispenser, and is dispensed from the at least one orifice to generate a plurality of microdroplets. The nanoparticle formation device is disposed to receive the microdroplets dispensed by the inkjet dispenser and form the nanoparticles therein.

### BACKGROUND

The present invention relates to a method and an apparatus for fabricating nanoparticles, and in particular, to a method and an apparatus for fabricating nanoparticles using an inkjet dispenser.

Nanotechnologies have advanced significantly during the last few years, and have been widely used in different areas such as the biochemical, medical and chemical industries. For example, nanotechnologies have allowed drugs to be delivered at enhanced rates due to increased surface area, thus enhancing adsorption rate and bioavailability. Furthermore, nanotechnologies have enabled water-insoluble drugs to be injected or absorbed, facilitating diagnosis and treatment. Nanotechnologies additionally provide great interest for cosmetics and tissue engineering scaffolds.

Current nanotechnologies commonly used in the preparation of controlled drug delivery are listed below.

Emulsion polymerization

Interfacial polymerization

Coagulated phase separation

Electrospray

Ultrasound

Supercritical fluid

Spray drying

Wet milling

Cryogenic technologies

Each of the above processes has its own advantages and limitations. The common limitation is that the size of generated nanoparticles is not uniform. For example, FIG. 1 is a schematic view of an ultrasonic atomizer assembly 10 contemplated for generation of nanoparticles, as disclosed in U.S. Pat. No. 6,767,637. The ultrasonic atomizer assembly 10 comprises an ultrasonic atomizer 11 and a collection bath 12. Two liquids comprising aqueous drug solution and the biodegradable polymer dissolved in organic solvents flow through the ultrasonic atomizer 11. As the ultrasonic atomizer 11 vibrates at an ultrasonic frequency, both liquids form a double layered film on the surface of the atomizer tip and are simultaneously fragmented into a large number of drops. Collision occurs among drops in proximity, which is followed by coalescence of the drops. A solvent-anti-solvent miscible process begins in the collection bath 12 as soon as the two microdrops come in contact. Since the liquids are randomly distributed, the size of generated nanoparticles is not uniform.

FIG. 2 is a schematic view of a supercritical fluid assisted nebulization and bubble drying system 20 as disclosed in U.S. Pat. No. 6,630,121. A liquid carbon dioxide is pumped by a supercritical carbon dioxide pump 21 from a carbon dioxide reservoir 22 via a conduit 23 through the pump 21 and via the conduit 23 to a mixing tee 24, where it becomes a supercritical fluid. A pump 25 pumps aqueous solvent from a solvent reservoir 26 via a conduit 27 where it is pumped via a conduit 28 to an injection port 29, where the drug of interest is added as an aqueous solution. The mixture in the mixing tee 24 expands downstream and forms aerosol A comprising fine particles of the substance dissolved or suspended in the aqueous solution. The particles are directed in the center of a drying tube 29a where they are dried and collected on a filter paper in a filter paper holder 29b. Since the mixture in the mixing tee 24 forms aerosol A without specific limitations, the size of generated nanoparticles is not uniform.

### SUMMARY

Apparatuses for fabricating nanoparticles are provided. An exemplary embodiment of an apparatus for fabricating nanoparticles comprises an inkjet dispenser and a nanoparticle formation device. The inkjet dispenser comprises at least one orifice. A liquid solution with a substance to be transformed into nanoscale is received in the inkjet dispenser, and is dispensed from the orifices to generate a plurality of microdroplets. The nanoparticle formation device is disposed to receive the microdroplets dispensed by the inkjet dispenser and form the nanoparticles therein.

The liquid solution is preferably composed of a solvent and a substance to be transformed into nanoscale dissolved therein. In an exemplary embodiment, the solvent is alcohol (Ethanol). It is understood that a mixture of solvents may also be employed.

Furthermore, the inkjet dispenser comprises a tank to receive the liquid solution, a nozzle plate on which the at least one orifice is formed, and an actuator for actuating the liquid solution to be dispensed. The actuator may be piezoelectric-type or thermal-type.

In a preferred embodiment, a liquid is received in the nanoparticle formation device, and nanoparticles are formed by solvent-anti-solvent miscible process between the microdroplets (solvent) and the liquid (anti-solvent). The liquid, or so-called anti-solvent, is one in which the substance to be transformed into nanoscale is insoluble. In exemplary embodiments, the anti-solvent is aqueous solution, which may contain certain solutes, or water only. The solvent used in the process is able to dissolve nanoparticle materials, while the anti-solvent is unable to dissolve the nanoparticle materials. In addition, the solvent is very miscible to the anti-solvent. The residual solvent or anti-solvent may be removed by further processes such as evaporation, dialysis, spray drying, vacuum evaporation or lyophilization.

In other embodiments, the nanoparticle formation device may include a freezing, extraction or heating drier for forming nanoparticles by freeze-drying, extraction-drying, or heat-drying in one process. In these embodiments, the nanoparticles comprise the substance to be transformed to nanoscale, where the solvent is removed by freezing-sublimation, drying or vacuum evaporation.

The apparatus may also comprise a separating device disposed between the inkjet dispenser and the nanoparticle formation device to separate the microdroplets according to size. The separating device may comprise a pair of deflection stations to separate the microdroplets, and a charging electrode to charge the microdroplets. In another embodiment, the separating device may comprise a blower to separate the microdroplets.

Substances suitable for transformation into nanoscale include bioactive material, polymer material, biomaterial, chemical material or mixtures thereof.

Methods for fabricating nanoparticles are also provided. An exemplary embodiment of a method for fabricating nanoparticles comprises the following steps. A liquid solution with a substance to be transformed into nanoscale is first placed in an inkjet dispenser. The inkjet dispenser is then actuated to dispense a plurality of microdroplets from the liquid solution. The nanoparticles are formed from the plurality of microdroplets.

The liquid solution is preferably composed of a solvent and the substance to be transformed to nanoscale dissolved therein. In an exemplary embodiment, the solvent is alcohol (Ethanol). The nanoparticles may be formed by solvent-anti-solvent process between the microdroplets (solvent) and a liquid (anti-solvent), wherein a preferred liquid is water. An aqueous solution may also be used. In other embodiments, the nanoparticles may be formed by removal of the solvent through heat-drying, extraction-drying or freezing-sublimation processes.

Additionally, the method may further comprise a step of directing the microdroplets in a first size range to travel along a first path and microdroplets in a second size range to travel along a second path after the microdroplets are dispensed, wherein the first and second paths diverge from the inkjet dispenser. The microdroplets may be directed by airflow, an electric field, or a magnetic field.

### DESCRIPTION OF THE DRAWINGS

The invention can be more fully understood by reading the subsequent detailed description and examples with references made to the accompanying drawings, wherein:

FIG. 1 is a schematic view of an ultrasonic atomizer assembly contemplated for generation of nanoparticles, as disclosed in U.S. Pat. No. 6,767,637;

FIG. 2 is a schematic view of a supercritical fluid assisted nebulization and bubble drying system as disclosed in U.S. Pat. No. 6,630,121;

FIG. 3 is a schematic view of an embodiment of an apparatus for fabricating nanoparticles;

FIGS. 4a and 4b are schematic views of an inkjet dispenser in FIG. 3;

FIG. 5 is a flowchart of an embodiment of a method for fabricating nanoparticles;

FIG. 6 is a schematic view of microdroplets generated by the inkjet dispenser in FIG. 3;

FIG. 7 is a schematic view of another embodiment of an apparatus for fabricating nanoparticles;

FIG. 8a is a schematic view of another embodiment of an apparatus for fabricating nanoparticles;

FIG. 8b is a schematic view of another embodiment of an apparatus for fabricating nanoparticles; and

FIG. 9 is a flowchart of an embodiment of a method for fabricating nanoparticles.

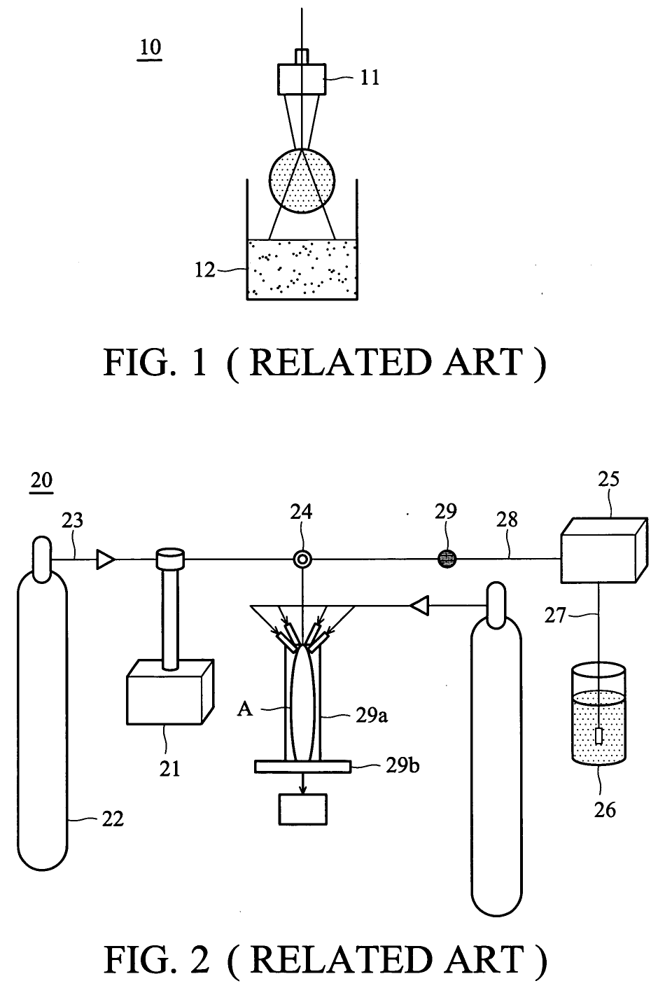

### DETAILED DESCRIPTION

The present invention provides apparatuses and methods for fabricating nanoparticles from a liquid solution with a substance to be transformed into nanoscale. The liquid solution is preferably composed of a solvent and a substance to be transformed into nanoscale dissolved therein. For example, a suitable solvent is alcohol (Ethanol). However, other solvents, or mixtures of solvents, which can dissolve the substance and are miscible with the anti-solvent selected in the nanoparticle formation device are also suitable. Substances suitable to be transformed into nanoscale include bioactive material, polymer material, biomaterial, chemical material or mixtures thereof. Note that the substances are active agents in the solvent. Furthermore, a stabilizer (excipient) may also be added in the solvent.

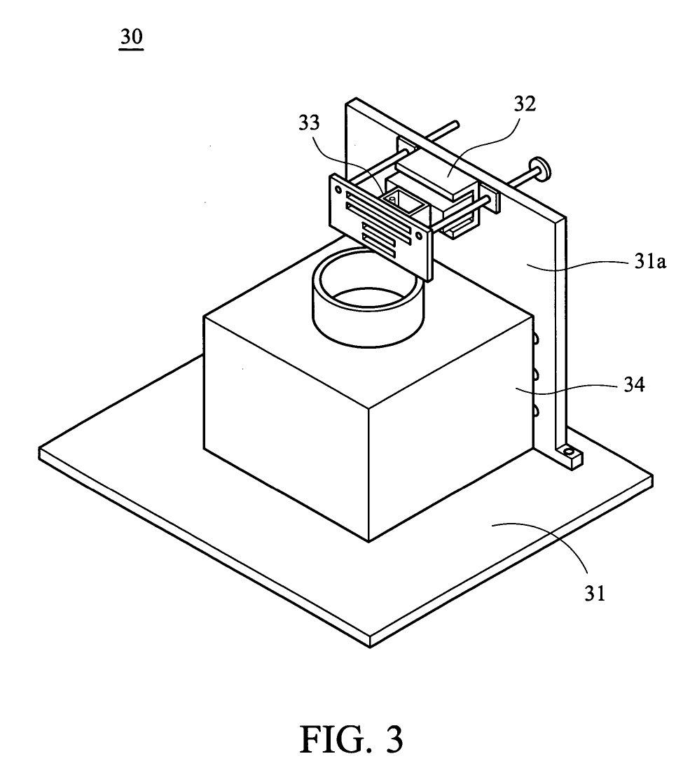

FIG. 3 is a schematic view of an embodiment of an apparatus 30 for fabricating nanoparticles. The apparatus 30 comprises a base 31, a holder 32, an inkjet dispenser 33, and a nanoparticle formation device 34. The holder 32 is disposed on an upright plate 31a of the base 31 to hold the inkjet dispenser 33.

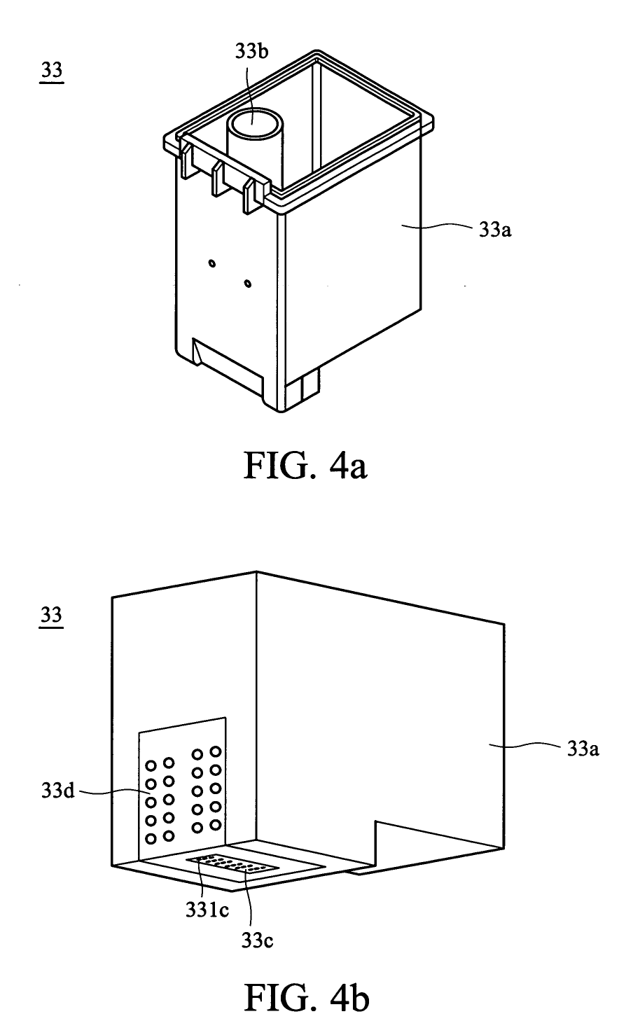

Referring to FIGS. 4a and 4b, the inkjet dispenser 33 comprises a body 33a, a tank 33b, a nozzle plate 33c, and a control circuit 33d. The tank 33b is disposed in the body 33a to receive the liquid solution. The nozzle plate 33c is disposed at the bottom of the body 33a, and comprises a plurality of orifices 331c. The liquid solution received in the tank 33b is dispensed from the orifices 331c to generate a plurality of microdroplets. The control circuit 33d is coupled to an external power source (not shown) and an actuator (not shown). The actuator may be piezoelectric-type or thermal-type to actuate the liquid solution to be dispensed.

Referring to FIG. 3, the nanoparticle formation device 34 is disposed on the base 31, and located below the inkjet dispenser 33. The nanoparticle formation device 34 fabricates the nanoparticles therein from the microdroplets dispensed by inkjet dispenser.

In a preferred embodiment, alcohol (Ethanol) serves as a solvent, which is able to dissolve nanoparticle materials to form the liquid solution. A liquid (water or aqueous solution) is received in the nanoparticle formation device 34 to serve as an anti-solvent, which is unable to dissolve the nanoparticle-forming materials. In addition, the solvent is miscible to the anti-solvent. Accordingly, when the microdroplets of alcohol containing liquid solution contact the water or aqueous solution, the solvent is quickly miscible with the anti-solvent. The substances, originally dissolved in the microdroplets, become insoluble in the mixture of solvent (alcohol) and anti-solvent (water or aqueous solution), and transform into solid nanoparticles. Thus, the active agents originally dissolved in the microdroplets become nanoscaled particles in the water or aqueous solution. Likewise, where a stabilizer (excipient) is dissolved in the liquid solution, the mixture of the active agents and the stabilizer (excipient) becomes nanoscaled particles in the water or aqueous solution. The residual solvent or anti-solvent may be removed by further processes such as evaporation, dialysis, spray drying or lyophilization.

In other embodiments, the nanoparticle formation device may include a freezing or heating drier for forming nanoparticles by freeze-drying, extraction-drying or heat drying in one process. In these embodiments, the nanoparticles comprise the substance to be transformed to nanoscale, where the solvent is removed by freezing-sublimation, extraction-drying or heat-drying.

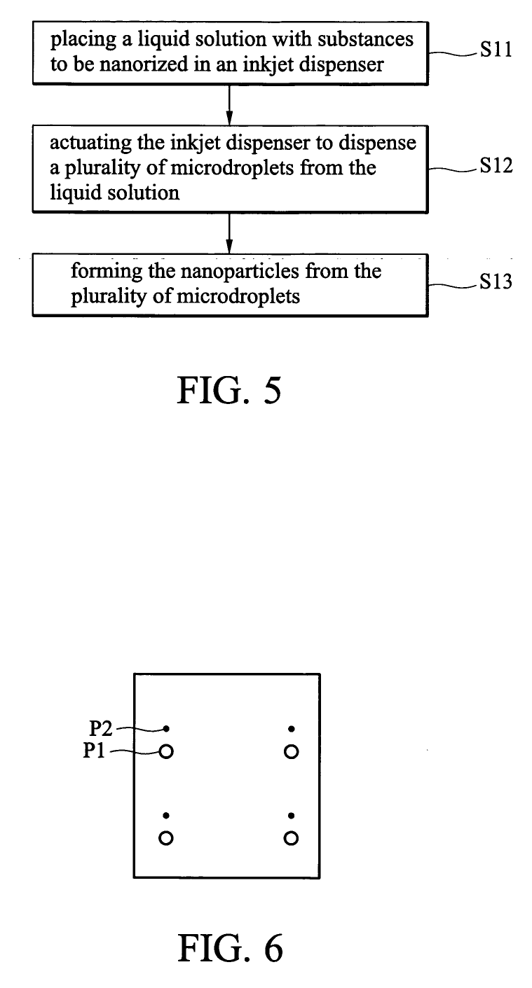

Referring to FIG. 5, an embodiment of a method for fabricating nanoparticles comprises the following steps. In step S11, the liquid solution with a substance to be transformed into nanoscale is first placed in the inkjet dispenser 33. In step S12, the inkjet dispenser 33 is then actuated to dispense a plurality of microdroplets from the liquid solution. In step S13, the nanoparticles are formed in the nanoparticle formation device 34 from the plurality of microdroplets.

Since the nanoparticles are generated by the microdroplets dispensed by the inkjet dispenser, their size can be precisely controlled, thus obtaining uniform nanoparticles.

### EXAMPLE 1

Phosphatidylcholine, a phospholipid, was dissolved in alcohol (Ethanol) to produce a solution of 2% Phosphatidylcholine by weight/volume. An inkjet dispenser with an orifice size of 30 μm and back pressure of 3 mbar dispensed microdroplets into a nanoparticle formation device containing DI water.

Nanoparticles with sizes in the range of 125.9˜199.5 nm (95.3 percent) and 12.6˜20.0 nm (4.7 percent) were produced when the variable control parameters were as follow:

1. voltage of inkjet dispenser: 15V

2. frequency of inkjet dispenser: 3 KHz

3. Pulse width of inkjet dispenser: 3.7 μs

4. working distance: 1 cm

where the working distance is the distance between the orifice of the inkjet dispenser and the water surface in the nanoparticle formation device.

The frequency of the inkjet dispenser is preferably not higher than 100 KHz. If the frequency is too high, a later dispensed microdroplet may catch up with a previously dispensed microdroplet to create an oversized microdroplet or a non-uniform distribution thereof. As a result, the microdroplet cannot be transformed into nanoscale, or the size of the nanoscaled particles is not uniform.

### EXAMPLE 2

An alcoholic mixture of 10% (w/v) ketoprophen, 0.4% (w/v) docusate sodium salt (DOSS) and 2% (w/v) Polyvinylpyrrolidone (PVP) was prepared by weight/volume to produce a solution. An inkjet dispenser with an orifice size of 30 μm and back pressure of 3 mbar dispensed microdroplets into a nanoparticle formation device containing DI water.

Nanoparticles sized in the range 158.5˜251.2 nm (100 percent) were produced when the variable control parameters were as follow:

1. voltage of inkjet dispenser: 15V

2. frequency of inkjet dispenser: 3 KHz

3. Pulse width of inkjet dispenser: 3.7 μs

4. working distance: 1 cm

where the working distance is the distance between the orifice of the inkjet dispenser and the water surface in the nanoparticle formation device.

With reference to FIG. 6, the dispensed microdroplets typically comprise major droplets P1 and minor droplets P2. As noted in Example 1, in a group of nanoparticles generated under the conditions described, the sizes of 95.3 percent of the nanoparticles range from 125.9 nm to 199.5 nm while the sizes of 4.7 percent range from 12.6 nm to 20.0 nm.

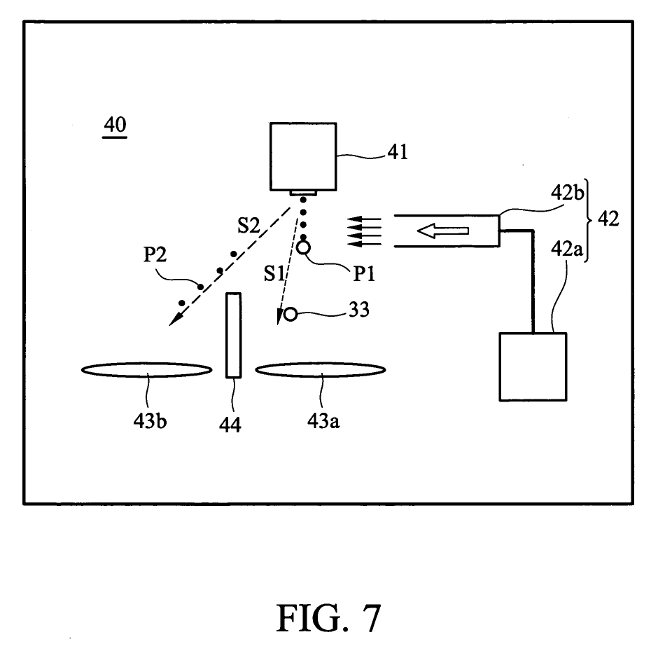

In view of this, another embodiment of an apparatus 40 for fabricating nanoparticles is provided. Referring to FIG. 7, the apparatus 40 comprises an inkjet dispenser 41, a separating device 42, two nanoparticle formation devices 43a and 43b, and an optional partition 44. Since the inkjet dispenser 41 is the same as the inkjet dispenser 33 in FIG. 3, its detailed description is omitted.

The separating device 42 is disposed above the nanoparticle formation devices 43a and 43b to separate the microdroplets dispensed from the inkjet dispenser 41 into major droplets P1 and minor droplets P2. The separating device 42 further directs major droplets P1 to nanoparticle formation device 43a and directs minor droplets P2 to nanoparticle formation device 43b. In FIG. 7, the separating device 42 comprises a gas source 42a and a blower 42b in communication with the gas source 42a. The blower 42b blows gas to the microdroplets to perform the separation.

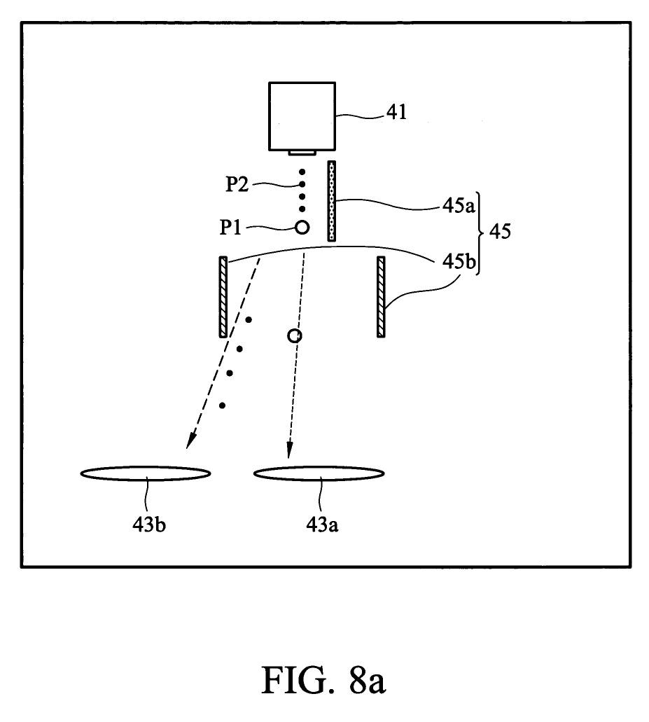

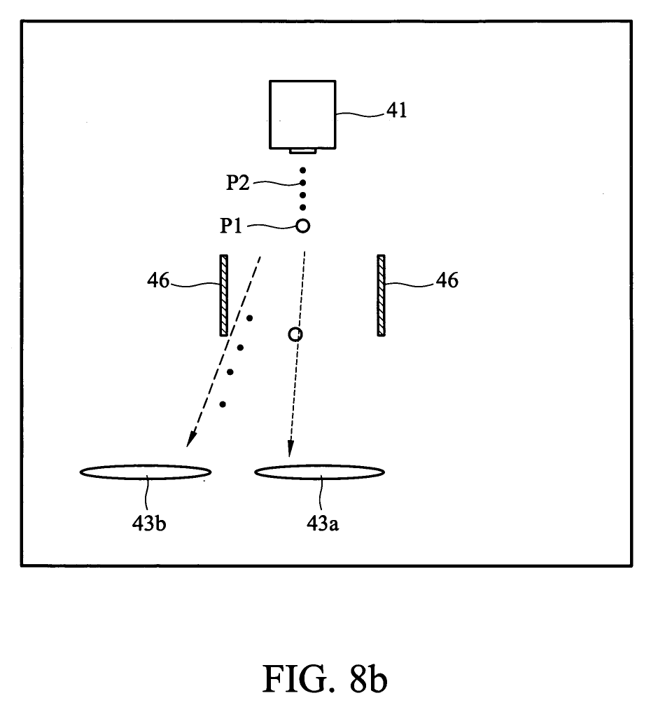

The separation device is not limited to the embodiment shown in FIG. 7. For example, as shown in FIG. 8a, in another embodiment the separating device 45 comprises a charging electrode 45a and a pair of deflection stations 45b. The charging electrode 45a charges the microdroplets. The deflection stations 45b separate the microdroplets according to size. Alternatively, if the microdroplets are already charged, the charging electrode can be omitted. That is, as shown in FIG. 8b, another embodiment of a separating device 46 simply comprises a pair of deflection stations to separate the microdroplets.

The nanoparticle formation device 43a fabricates the nanoparticles therein from the major droplets P1, and the nanoparticle formation device 43b fabricates the nanoparticles therein from the minor droplets P2. The optional partition 44 may be disposed between the nanoparticle formation device 43a and 43b to prevent the major droplets from traveling into the nanoparticle formation device 43b. The partition may also be omitted, as shown in FIGS. 8a and 8b.

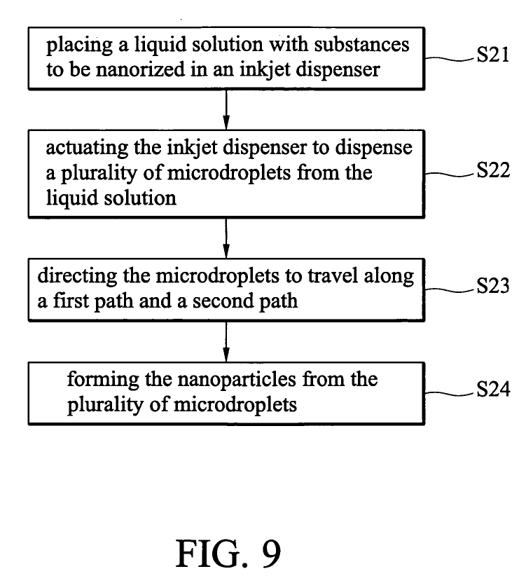

Referring to FIG. 9, another embodiment of a method for fabricating nanoparticles comprises the following steps. In step S21, a liquid solution with a substance to be transformed into nanoscale is first placed in the inkjet dispenser 41. In step S22, the inkjet dispenser 41 is then actuated to dispense a plurality of microdroplets from the liquid solution. In step S23, the microdroplets of a first size range are directed to travel along a first path S1 and microdroplets of a second size range to travel along a second path S2 as shown in FIG. 7, wherein the first and second paths S1 and S2 diverge from the inkjet dispenser 41. In step S24, nanoparticles of a first size range are formed from microdroplets traveling along the first path in the nanoparticle formation device 43a, while other nanoparticles of a second size range are formed from microdroplets traveling along the second path in the nanoparticle formation devices 43b.

Although the microdroplets are directed by airflow generated by the blower 42b in FIG. 7 or an electric field generated by the deflection plates 45b and 46 in FIGS. 8a and 8b, they may also be directed by a magnetic field.

Since the microdroplets from the inkjet dispenser are further separated by the separation device according to size in this embodiment, the uniformity of the size of the nanoparticles can be enhanced.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An apparatus for fabricating nanoparticles from a liquid solution with a substance to be transformed into nanoscale, comprising:
an inkjet dispenser, comprising at least one orifice, for receiving the liquid solution and dispensing a plurality of microdroplets; and
a nanoparticle formation device disposed to receive the microdroplets and form the nanoparticles there from.

2. The apparatus as claimed in claim 1, wherein
the liquid solution is composed of a solvent and the substance to be transformed to nanoscale dissolved therein.

3. The apparatus as claimed in claim 2, wherein the solvent is alcohol.

4. The apparatus as claimed in claim 2, wherein the nanoparticle formation device contains a liquid, and the nanoparticles are generated due to solvent-anti-solvent miscible process between the microdroplets and the liquid.

5. The apparatus as claimed in claim 4, wherein the liquid is water or aqueous solution.

6. The apparatus as claimed in claim 4, wherein the liquid is one in which the substance to be transformed to nanoscale is insoluble.

7. The apparatus as claimed in claim 1, wherein the nanoparticles formation device works by a process to remove the solvent from the liquid solution.

8. The apparatus as claimed in claim 7, wherein the process is selected from the group consisting of heat-drying, extraction-drying, freezing-sublimation, vacuum evaporation and solvent exchanging process.

9. The apparatus as claimed in claim 1, wherein the substance to be transformed into nanoscale is bioactive material, polymer material, biomaterial or a mixture thereof.

10. The apparatus as claimed in claim 1, wherein the frequency of the inkjet dispenser is not larger than 100 KHz.

11. An apparatus for fabricating nanoparticles from a liquid solution with a substance to be transformed into nanoscale, comprising:
an inkjet dispenser comprising at least one orifice for receiving the liquid solution and dispensing a plurality of microdroplets;
a first nanoparticle formation device for forming nanoparticles from microdroplets received thereby; and
a separating device disposed between the inkjet dispenser and the first nanoparticle formation devices to separate the microdroplets into a first group of microdroplets in a first size range and a second group of microdroplets in a second size range, and direct the first group of microdroplets to the first nanoparticle formation device.

12. The apparatus as claimed in claim 11, wherein the separating device comprises a pair of deflection stations to separate the microdroplets according to size.

13. The apparatus as claimed in claim 12, wherein the separating device further comprises a charging electrode to charge the microdroplets.

14. The apparatus as claimed in claim 11, wherein the nanoparticles formation device works by a process to remove the solvent from the liquid solution.

15. The apparatus as claimed in claim 14, wherein the process is selected from the group consisting of heat-drying, extraction-drying, freezing-sublimation, vacuum evaporation and solvent exchanging process.

16. The apparatus as claimed in claim 11, further including a second nanoparticle formation device for forming nanoparticles from microdroplets received thereby, wherein the separating device directs the second group of microdroplets to the second nanoparticle formation device.

17. The apparatus as claimed in claim 11, wherein the frequency of the inkjet dispenser is not larger than 100 KHz.

18. A method for fabricating nanoparticles, comprising:
placing a liquid solution with a substance to be transformed into nanoscale in an inkjet dispenser;
actuating the inkjet dispenser to dispense a plurality of microdroplets of the liquid solution; and
forming the nanoparticles from the plurality of microdroplets.

19. The method as claimed in claim 18, wherein the liquid solution is composed of a solvent and the substance to be transformed to nanoscale dissolved therein.

20. The method as claimed in claim 19, wherein the solvent is alcohol.

21. The method as claimed in claim 19, wherein the nanoparticles are formed by solvent-anti-solvent miscible process between the microdroplets and a liquid.

22. The method as claimed in claim 21, wherein the liquid is water or aqueous solution.

23. The method as claimed in claim 21, wherein the liquid is one in which substance to be transformed to nanoscale is insoluble, but is miscible with the solvent.

24. The method as claimed in claim 18, wherein the substance to be transformed into nanoscale is bioactive material, polymer material, biomaterial or a mixture thereof.

25. The method as claimed in claim 18, further comprising:
directing microdroplets of a first size range to travel along a first path and directing microdroplets of a second size range to travel along a second path after the microdroplets are dispensed, wherein the first and second paths diverge from the inkjet dispenser.

26. The method as claimed in claim 25, wherein the microdroplets are directed by airflow, an electric field, or a magnetic field.
